(19) United States
(12) Patent Application Publication
(10) Pub. No.: US 2025/0383271 A1
(43) Pub. Date: Dec. 18, 2025

(54) Analytical Pretreatment Method

(71) Applicants: NATIONAL INSTITUTE OF ADVANCED INDUSTRIAL SCIENCE AND TECHNOLOGY, Tokyo (JP); SHIMADZU CORPORATION, Kyoto-shi (JP)

(72) Inventors: Daisuke MIURA, Tsukuba-shi (JP); Naohiro NODA, Tsukuba-shi (JP); Masamune MORITA, Tsukuba-shi (JP); Ryogo TAKAI, Kyoto-shi (JP)

(73) Assignees: NATIONAL INSTITUTE OF ADVANCED INDUSTRIAL SCIENCE AND TECHNOLOGY, Tokyo (JP); SHIMADZU CORPORATION, Kyoto-shi (JP)

(21) Appl. No.: 19/236,577

(22) Filed: Jun. 12, 2025

(30) Foreign Application Priority Data
Jun. 13, 2024 (JP) 2024-095998

(51) Int. Cl. G01N 1/38; G01N 1/40; G01N 33/68

(52) U.S. Cl. CPC G01N 1/38; G01N 1/4022; G01N 33/6851; G01N 2001/4027

(57) ABSTRACT

An analytical pretreatment method for analysis of samples includes: preparing an emulsion including oil, and a first droplet and a second droplet being present in the oil and containing a first sample and a second sample respectively; placing, on a substrate, an aggregate of the first droplet and the second droplet in the emulsion; and evaporating the oil and water on the substrate to separate a first evaporation residue and a second evaporation residue from each other that include the first sample and the second sample respectively.

### CROSS REFERENCE TO RELATED APPLICATIONS

This nonprovisional application is based on Japanese Patent Application No. 2024-095998 filed on Jun. 13, 2024 with the Japan Patent Office, the entire contents of which are hereby incorporated by reference.

### BACKGROUND OF THE INVENTION

### Field of the Invention

The present disclosure relates to an analytical pretreatment method, and more specifically relates to a pretreatment method for analyzing samples in droplets.

### Description of the Background Art

As one of the cell culture methods, there has been a technique for culturing cells in a state in which water droplets (droplets) enclosing a culture medium containing the cells are dispersed in oil. According to the technique, the droplets are used as independent incubators separated by the oil.

Sara E. Bell et al., “Droplet Microfluidics with MALDI-MS Detection: The Effects of Oil Phases in GABA Analysis”, ACS Measurement Science, 2021, 1, 3, pp. 147-156., https://doi.org/10.1021/acsmeasuresciau.1c00017 discloses a technique for regularly depositing droplets one by one onto a glass slide by means of three-axis micromanipulators.

### SUMMARY OF THE INVENTION

The technique of the above-referenced document may be utilized as a pretreatment method for sample analysis, which, however, still has a room for improvements in terms of the throughput.

An object of the present invention is to provide a high-throughput method for fixing samples in respective droplets in a state where the samples are separated from each other.

An analytical pretreatment method according to an aspect of the present disclosure is an analytical pretreatment method for analysis of samples, including: preparing an emulsion including oil, and a first droplet and a second droplet being present in the oil and containing a first sample and a second sample respectively; placing, on a substrate, an aggregate of the first droplet and the second droplet in the emulsion; and evaporating the oil and water on the substrate to separate a first evaporation residue and a second evaporation residue from each other that include the first sample and the second sample respectively.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a schematic configuration diagram of an analytical system according to an embodiment.

FIG. 2 is a diagram for illustrating pretreatment according to an embodiment.

FIG. 3 is a diagram for illustrating the pretreatment according to the embodiment.

FIG. 4 is a flowchart showing an example of a pretreatment method according to an embodiment.

FIG. 5 is a diagram showing droplets while being dried, with different surfactant concentrations.

FIG. 6 is a diagram showing mass spectra with different surfactant concentrations.

FIG. 7 is a diagram showing evaporation residues under different humidity conditions.

FIG. 8 is a diagram for illustrating pretreatment according to a modification.

FIG. 9 is a flowchart showing an example of a pretreatment method according to the modification.

FIG. 10 is a diagram illustrating withdrawal of samples according to an example.

FIG. 11 is a diagram illustrating results of analysis according to an example.

FIG. 12 is a diagram illustrating results of analysis according to an example.

### DESCRIPTION OF THE PREFERRED EMBODIMENTS

Embodiments of the present disclosure are hereinafter described in detail with reference to the drawings. In the drawings, the same or corresponding parts are denoted by the same reference characters, and a description thereof is not herein repeated.

### [Configuration of Analytical System]

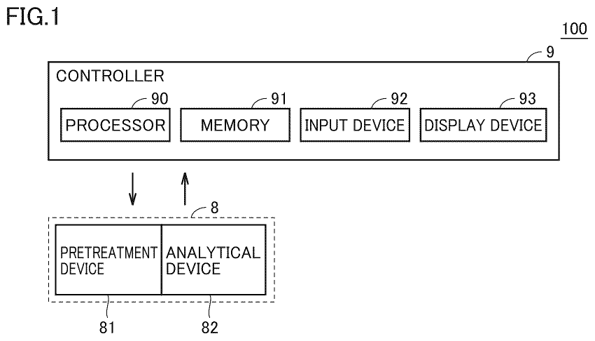

FIG. 1 is a schematic configuration diagram of an analytical system 100 according to the present embodiment.

Analytical system 100 includes an experimental device 8 and a controller 9. Experimental device 8 includes a pretreatment device 81 and an analytical device 82.

Pretreatment device 81 performs a pretreatment method for analysis of samples according to the present embodiment. The pretreatment method for analysis of samples is also referred to herein as “analytical pretreatment method.”

Analytical device 82 analyzes samples pretreated by pretreatment device 81. According to one example, analytical device 82 is a mass spectrometer. In a more specific example, analytical device 82 is a mass spectrometer conducting MALDI analysis.

Controller 9 controls pretreatment device 81 and analytical device 82. Controller 9 may monitor the process and/or the results of pretreatment by pretreatment device 81, and may analyze the process and/or the results of analysis by analytical device 82.

Controller 9 includes a processor 90, a memory 91, an input device 92, and a display device 93.

Processor 90 includes a CPU (Central Processing Unit), for example. Processor 90 deploys a program stored in memory 91, into a RAM or the like, and executes the program.

Memory 91 includes a ROM (Read Only Memory), a RAM (Random Access Memory), and a nonvolatile memory, for example. A program stored in the ROM is a program in which a process procedure of controller 9 is specified. The nonvolatile memory stores data sent from pretreatment device 81 and/or analytical device 82. Memory 91 may include a hard disk device instead of or in addition to the nonvolatile memory.

Input device 92 is a device for inputting a user's instruction to controller 9. For example, input device 92 includes a keyboard and a pointing device such as mouse.

Display device 93 includes a liquid crystal display or the like. Display device 93 may display the process and/or the results of pretreatment by pretreatment device 81, and may display the process and/or the results of analysis by analytical device 82.

### Pretreatment Method According to Embodiment

A pretreatment method according to an embodiment may be performed by controller 9 controlling pretreatment device 81, or may be performed manually by a user.

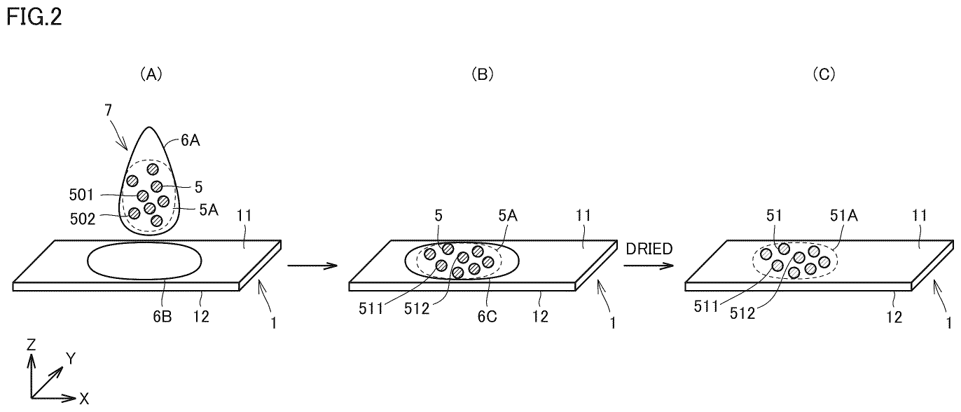

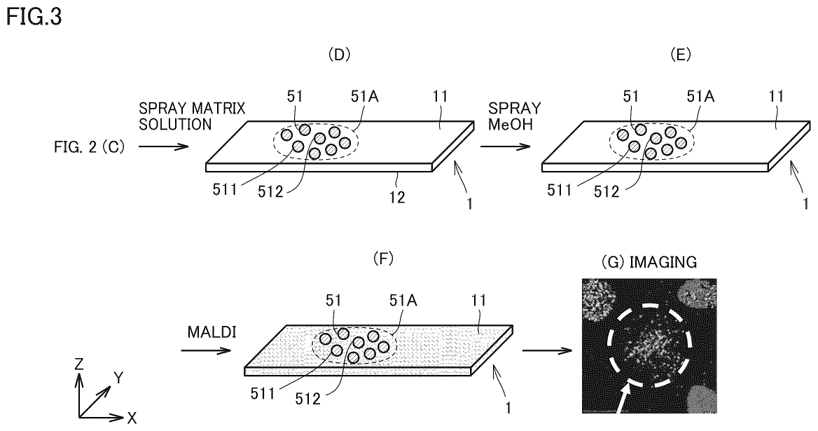

FIGS. 2 and 3 are diagrams for illustrating pretreatment according to an embodiment. Referring to FIG. 2, oil 6 may be referred to herein differently as appropriate, as “first oil 6A which is the oil before being placed on substrate 1 and in which droplets 5 are dispersed,” “second oil 6B placed in advance on substrate 1,” and “third oil 6C which is the oil after being placed on substrate 1 and in which droplets 5 are dispersed.” “Droplets 5 are dispersed in oil 6” herein does not necessarily mean that droplets 5 in oil 6 are located apart from each other, which means that at least some of droplets 5 may be in contact with each other. In FIG. 2, (A) to (C) indicate respective steps common to pretreatment methods according to embodiments. In FIG. 3, (D) to (E) indicate respective steps peculiar to pretreatment for MALDI analysis. In FIG. 3, (F) to (G) indicate respective steps of MALDI analysis and MALDI imaging. In other words, (A) to (E) in FIGS. 2 and 3 indicate a pretreatment method for MALDI analysis, which is one example of the pretreatment methods according to embodiments.

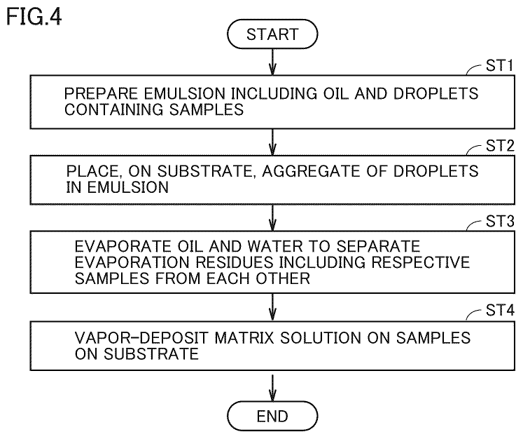

FIG. 4 is a flowchart showing an example of the pretreatment method according to an embodiment. Each step (also referred to as “ST” hereinafter) in FIG. 4 may be performed by controller 9 controlling pretreatment device 81, or may be performed manually by an analyst. The pretreatment method according to an embodiment is hereinafter described following FIG. 4, with reference to FIGS. 2 and 3 as appropriate.

In ST1 of FIG. 4, an emulsion 7 is prepared. Emulsion 7 includes oil 6, and droplets 5 that are present in oil 6 and contain respective samples. More specifically, emulsion 7 is prepared that includes oil 6, and a first droplet 501 and a second droplet 502 that are present in oil 6 and contain a first sample and a second sample respectively.

In one example, the sample includes a cell and/or a cell-derived substance. The cell is made up of metabolites of protein, lipid, nucleic acid, and carbohydrate, for example. The cell-derived substance herein typically includes metabolites released from the cell, and may also include a part of the cell having been broken. Therefore, by analyzing components included in a droplet 5, the cell itself (cell body, for example) or metabolites located inside and outside the cell can be analyzed. In a typical example, droplet 5 also contains a culture medium for the cell including the sample. In this way, droplet 5 can be used as an incubator for a specific cell.

Emulsion 7 is a water-in-oil emulsion. Emulsion 7 is generated, for example, by generating, in oil 6, droplets 5 including a culture medium including cells, by means of a microfluidic channel device.

Droplets 5 are generated by making adjustments to allow each droplet 5 to preferably include one or zero cell. Thus, each droplet 5 includes a sample derived from a single cell. In other words, each droplet 5 includes only one type of cell and a substance derived from the cell. Accordingly, analysis of each sample is facilitated, relative to the case where each droplet 5 includes multiple types of cells, and in particular, the analysis is further facilitated relative to the case where each droplet includes many cells.

For example, in the case where it is found that the rate of increase of a predetermined metabolite is high in a certain droplet 5 and this droplet 5 includes multiple types of cells, it is difficult to identify a specific type of cell from which the metabolite whose rate of increase is high is derived. In this case, this droplet 5 also includes genes involved in generation of the metabolite, where the number of the genes is identical to the number of the types of the cells, and therefore, it is also difficult to identify a gene that causes the high rate of increase of the metabolite. These difficulties are higher in the case where droplet 5 includes many types of cells, relative to the case where droplet 5 includes a few types of cells. In view of this, preferably each droplet 5 includes as fewer types of cells as possible, and more specifically includes only one type of cell. It is also preferable that the samples are fixed under a condition that a plurality of droplets 5 are not fused together while the samples are fixed. Genes herein refer to a DNA (deoxyribonucleic acid) sequence and/or an RNA (ribonucleic acid) sequence.

In one example, the first sample and the second sample contained respectively in first droplet 501 and second droplet 502 are samples of the same type. More specifically, the first sample and the second sample contained respectively in first droplet 501 and second droplet 502 are samples derived from cells of the same type. In this way, it is possible to analyze each of the first and second samples to thereby analyze how each of the multiple types of samples derived from cells of the same type is changed. Likewise, in the case where the first and second samples are samples of the same strain, it is possible to analyze how each of the multiple types of samples derived from cells of the same strain is changed.

In ST2, an aggregate 5A of droplets 5 in emulsion 7 is placed on a substrate 1. More specifically, aggregate 5A of first droplet 501 and second droplet 502 in emulsion 7 is placed on the substrate. “Aggregate 5A of first droplet 501 and second droplet 502” herein refers to aggregate 5A of a plurality of droplets including first droplet 501 and second droplet 502. Substrate 1 has a surface 11 on which aggregate 5A of droplets 5 is to be placed. Surface 11 is formed to be flat to facilitate analysis of a plurality of droplets 5 on surface 11 at a time. In one example, substrate 1 is a substrate having surface 11 and a surface 12 that are two flat surfaces opposite to each other.

In a more specific example, substrate 1 is a sample plate for mass spectrometry using MALDI method. The mass spectrometry using MALDI method is also referred to herein as “MALDI analysis” and the sample plate for the MALDI analysis is also referred to herein as “MALDI plate.” In the case where the MALDI plate is used as substrate 1, the pretreatment method according to the present embodiment is performed on samples on substrate 1 and thereafter substrate 1 is moved to a mass spectrometer, to thereby enable the MALDI analysis to be conducted simply and conveniently. In particular, MALDI imaging, which is described later herein, can be conducted to thereby enable high-throughput analysis of each of samples on substrate 1.

A plane parallel to substrate 1 may be referred to herein as XY plane and the direction orthogonal to the XY plane may be referred to herein as Z-axis direction. Moreover, the positive Z-axis direction (the direction from surface 12 toward surface 11 of substrate 1) may be referred to herein as “upward” and the negative Z-axis direction (the direction from surface 11 toward surface 12 of substrate 1) may be referred to herein as “downward.”

Aggregate 5A herein includes a plurality of droplets 5. In other words, aggregate 5A includes a collection of droplets 5. Aggregate 5A corresponds to one example of “aggregate.”

In one example of ST2, aggregate 5A is dropped on surface 11. In another example, aggregate 5A may be spooned with a spoon and placed on surface 11. Aggregate 5A may also be placed on surface 11 with a syringe or the like having its tip to be brought into contact with surface 11.

In one example, droplets 5 are generated in oil having a surfactant concentration of about 2%. In oil having a lower surfactant concentration, droplets 5 are unstable. Therefore, for fixation with a lower surfactant concentration, second oil 6B of a low concentration is placed in advance on a portion of surface 11 where aggregate 5A is to be placed and then droplets 5 in the oil having a surfactant concentration of about 2% are dropped in second oil 6B to thereby enable droplets 5 to be held stably until right before the fixation. While second oil 6B may be placed in advance on the portion of surface 11 where aggregate 5A is to be placed as described above, second oil 6B may not be placed, in another example, on the portion of surface 11 where aggregate 5A is to be dropped. In this way, it is possible to save the need of placing second oil 6B in advance on surface 11.

In ST3, oil 6 (third oil 6C in FIG. 2) and water on substrate 1 are evaporated to separate evaporation residues 51 including respective samples from each other. More specifically, oil 6 and water on substrate 1 are evaporated to separate a first evaporation residue 511 and a second evaporation residue 512 from each other that include the first sample and the second sample respectively. First evaporation residue 511 is a product generated by drying first droplet 501 and second evaporation residue 512 is a product generated by drying second droplet 502. The water is water included in the culture medium in droplets 5, and/or water included in the cells, for example. In the example in FIG. 2, third oil 6C is an oil mixture of first oil 6A and second oil 6B. In an example where second oil 6B is not placed in advance on substrate 1, third oil 6C is the same oil as first oil 6A.

By ST3, oil 6 and water are removed from substrate 1 and droplets 5 placed on substrate 1 become evaporation residues 51. Generating evaporation residues 51 in a state where samples can be analyzed, by drying oil 6 in emulsion 7 and the water is also referred to herein as “fixing/fixation.” It should be noted that evaporation residues 51 may include oil 6, a surfactant and/or water to the extent that does not hinder analysis of the samples.

The collection of evaporation residues 51 including a plurality of evaporation residues 51 is referred to herein as an evaporation residue aggregate 51A.

In ST4, a matrix solution is vapor-deposited on samples on substrate 1. More specifically, the matrix solution is vapor-deposited on the first sample and the second sample on substrate 1. ST4 is performed after ST3.

The matrix solution is a solution containing a matrix substance. The matrix substance is a substance that easily absorbs laser energy to easily ionize a compound included in an analysis sample. The matrix substance is α-Cyano-4-Hydroxycinnamic acid (4-CHCA), 2,5-dihydroxybenzoic acid (DHB), or sinapinic acid (SA), for example.

In one example of ST4, the matrix solution is sprayed toward evaporation residues 51 on the MALDI plate. By spraying the matrix solution, the matrix solution is vapor-deposited on the samples in evaporation residues 51. In this way, the matrix solution can be vapor-deposited easily and conveniently on each of the samples, even when the samples are dispersed irregularly on the MALDI plate.

The samples having undergone the pretreatment in FIG. 3 can be subjected to MALDI analysis. In one example, MALDI analysis is performed by scanning a laser beam across the whole of evaporation residue aggregate 51A on substrate 1, to acquire the results of the analysis of the whole of evaporation residue aggregate 51A at a time, and thereafter MALDI imaging is performed to image the results of the MALDI analysis.

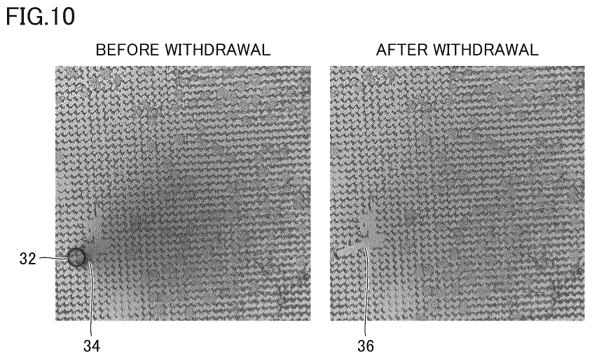

The mesh patten on substrate 1 in (F) of FIG. 3 indicates a trace of the laser beam applied during the MALDI analysis (see FIG. 10). Regarding the MALDI imaging, the color (the brightness in (G) of FIG. 3) indicates the strength of the signal intensity. Thus, a distribution of the strength of the signal intensity on substrate 1 can be expressed in the form of an image. Accordingly, a distribution of a substance with a predetermined m/z can be identified visually, as a distribution of a color indicating the signal intensity at the m/z.

Thus, the MALDI imaging enables the results of mass spectrometry of respective samples placed at respective positions on substrate 1 to be acquired at a time. Accordingly, components contained in each sample can be analyzed with a high throughput.

As shown exemplarily in FIG. 3, methanol (MeOH) may be sprayed after the matrix solution is sprayed and before the MALDI analysis is performed.

Thus, according to the embodiment, it is possible to fix, on substrate 1, samples derived from respective droplets 5, without the step of isolating droplets 5 from each other, without the step of placing each droplet 5 at a predetermined position on substrate 1, and without causing mixture of the samples derived from respective droplets 5. In other words, it is possible to provide a high-throughput method for fixing samples in respective droplets in a state where these samples are separated from each other. It is therefore possible to improve the throughput for fixing samples in respective droplets 5 in emulsion 7, as compared with conventional methods. Accordingly, it is also possible to improve the throughput of the whole analysis of samples included in respective droplets 5 in emulsion 7.

In the process of FIG. 4, the samples included in respective droplets 5 may be microorganisms, and the process of FIG. 4 may further include the step of culturing the microorganisms in emulsion 7. More specifically, in the process of FIG. 4, the first sample and the second sample included respectively in droplets 501 and 502 are microorganisms, and the process of FIG. 4 may further include the step of culturing the microorganisms in emulsion 7. In this way, it is possible to grow, to a sufficient extent, the microorganisms themselves in respective droplets 5 and/or a predetermined metabolite generated from the microorganisms, and then perform analysis on them. The sufficient extent is an extent suitable for analysis.

In one example, aggregate 5A is generated by a microfluidic channel device, then cultured in a predetermined container for a predetermined time, and thereafter placed on substrate 1. In another example, aggregate 5A may be placed on substrate 1 immediately after being generated by a microfluidic channel device, and then cultured on substrate 1. In the case where the step of culturing prior to the analysis is unnecessary, aggregate 5A may be analyzed immediately after being placed on substrate 1 immediately after being generated by a microfluidic channel device.

It is also possible to manage the samples on substrate 1 in association with respective positions (XY coordinates, for example) on substrate 1. The traceability of each sample is thus ensured, and therefore, it is also possible to perform another analysis such as gene analysis on any sample on substrate 1 focused on by the MALDI analysis (see a modification described later herein).

In one example, the volatility of oil 6 is equivalent to or higher than the volatility of water. Accordingly, in ST3, oil 6 can be evaporated at a speed equivalent to or higher than the speed at which water is evaporated. As a result, it is possible to prevent droplets 5 from being deformed due to oil 6, while water is evaporated. Moreover, at the time water has been evaporated completely, oil 6 has also been evaporated completely, and therefore, it does not occur that droplets 5 are deformed due to remaining oil 6. The aforementioned “oil higher in volatility than water” is fluorine oil, for example. In some cases, fluorine oil is also called fluorine-based oil by those skilled in the art.

In the case where oil larger in specific gravity than water is used as oil 6, droplets 5 float up to the surface of emulsion 7 in ST3, so that emulsion 7 can be dried while droplets 5 are present in a single layer, without overlapping each other. As a result, droplets 5 are less likely to be fused together while being dried, as compared with the case where oil smaller in specific gravity than water is used and emulsion is dried while droplets sink within the emulsion. In this respect as well, it is preferable to use, as oil 6, fluorine oil larger in specific gravity than water.

Next, environmental conditions under which ST3 (fixing step) is performed are described in detail. The inventors have examined various conditions to find that the surfactant concentration in oil 6 and the humidity around substrate 1 are important for preventing droplets 5 from being fused with each other. In view of this, environmental conditions under which droplets 5 are not fused with each other herein include a concentration condition that the concentration of a surfactant in oil 6 on substrate 1 during the fixation (ST3) is within a predetermined concentration range, and/or a humidity condition that the humidity around substrate 1 during the fixation (ST3) is within a predetermined humidity range. As described below, the concentration condition and/or the humidity condition can be adjusted to thereby generate evaporation residues 51 that can be analyzed and/or evaporation residues 51 suitable for analysis.

The temperature around substrate 1 in ST3 is preferably room temperature. The room temperature is 20° C. to 25° C., for example.

### [Surfactant Concentration Condition]

As the surfactant used in the present embodiment, a surfactant is preferable that is easily mixed with fluorine oil and allows droplets 5 mixed with fluorine oil to be easily held stably. In this respect, it is preferable for the present embodiment to use a fluorine-based surfactant. As the fluorine-based surfactant, for example, commercially available 008-FluoroSurfactant (RAN Biotechnologies), or Pico-Surf® (Sphere Fluidics) is used.

The concentration of the surfactant is, for example, 1% by mass or more and 4.5% by mass or less, and preferably 1% by mass or more and 2% by mass or less, however, the surfactant concentration is not limited to the above-specified ones. In this way, evaporation residues 51 that can be analyzed, and/or evaporation residues 51 suitable for analysis are generated, as illustrated exemplarily in the following.

In the case where the concentration of the surfactant in oil 6 is indicated herein with “%” only, this means mass % (w/w %) unless otherwise specified.

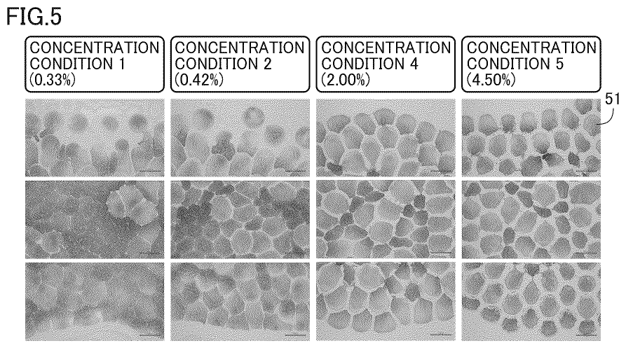

FIG. 5 is a diagram showing droplets 5 while being dried, with different surfactant concentrations. FIG. 5 shows droplets 5 while being dried under concentration conditions 1, 2, 4, and 5. Concentration conditions 1 to 5 herein correspond respectively to conditions: the amount of first oil 6A in which droplets 5 before being placed on substrate 1 are dispersed is 1 μL, the concentration of the surfactant in first oil 6A is 2%, the amount of second oil 6B placed in advance on substrate is 5 μL, and respective concentrations of the surfactant in second oil 6B are 0%, 0.1%, 1%, 2%, and 5%.

The concentration of third oil 6C after droplets 5 are placed on substrate is determined by: {(amount of first oil)×(concentration of surfactant in first oil)+ (amount of second oil)×(concentration of surfactant in second oil)}/{(amount of first oil)+ (amount of second oil)}. Therefore, under the concentration conditions 1 to 5, the concentrations of the surfactant in third oil 6C are 0.33%, 0.42%, 1.17%, 2.00%, and 4.50%, respectively.

Other environmental conditions included conditions that the humidity of substrate 1 was about 30% and the temperature thereof was room temperature. As first oil 6A, 2% (w/w) 008-FluoroSurfactant (RAN Biotechnologies) was used. As second oil 6B of concentration conditions 2 to 5, oil prepared by diluting 2% (w/w) 008-FluoroSurfactant (RAN Biotechnologies) with fluorine oil as appropriate was used. As second oil 6B of concentration condition 1, fluorine oil was used.

Referring to FIG. 5, it is seen that, as the concentration of the surfactant in oil 6 (third oil 6C) on substrate 1 is higher, droplets 5 do not overlap each other on the XY plane and the distance between droplets 5 is larger. Under the concentration conditions 1 and 2, droplets 5 were fused with each other in some cases. In contrast, under the concentration conditions 4 and 5, droplets 5 were not fused with each other. Under the concentration condition 3 as well, droplets 5 were not fused with each other. Therefore, in order to prevent droplet 5 from being fused with each other, the surfactant concentration in oil 6 on substrate 1 is preferably higher than the concentration condition 3 (about 1%). In order to increase the distance between droplets 5, it is preferable that the concentration of the surfactant in oil 6 on substrate 1 is higher. For example, referring to FIG. 5, the gap between droplets 5 is larger under the concentration condition 5 than that under the concentration condition 4, and therefore, the concentration condition 5 is more preferable.

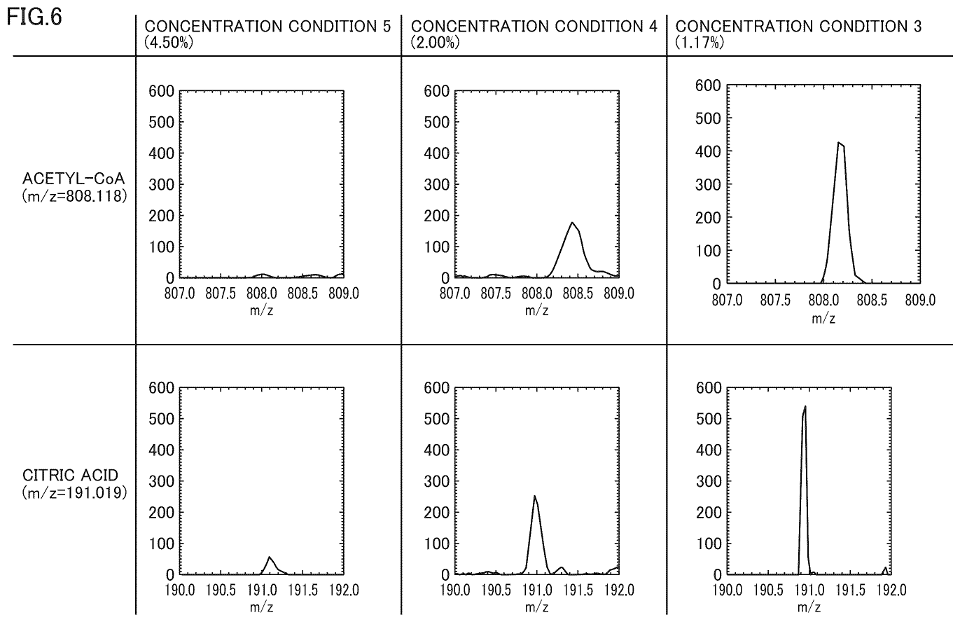

FIG. 6 is a diagram showing mass spectra with different surfactant concentrations. FIG. 6 shows mass spectra of samples pretreated under the concentration conditions 3 to 5. The upper row of FIG. 6 shows mass spectra around m/z=808.118 associated with acetyl-CoA. The lower row of FIG. 6 shows mass spectra around m/z=191.019 associated with citric acid. Referring to FIG. 6, in the mass spectra under the concentration condition 5, a peak at or near m/z=808.118 and a peak at or near m/z=191.019 are detected, however, respective intensities of the peaks are relatively small and, in particular, the peak at or near m/z=808.118 is not clearly distinguished from the signal intensity around the peak. In contrast, in the mass spectra under the concentration conditions 3 and 4, a peak at or near m/z=808.118 and a peak at or near m/z=191.019 are clearly detected. In the mass spectra under the concentration condition 3, the detected peaks are higher than those in the mass spectra under the concentration condition 4. According to FIG. 6, it is preferable that the concentration of the surfactant in oil 6 on substrate 1 is lower. This is presumably because a higher concentration of the surfactant in oil 6 on substrate 1 leads to a larger amount of the surfactant contained in evaporation residue 51, which hinders mass spectrometry. For example, presumably a phenomenon occurs that a laser beam is incident on the surfactant, which hinders the laser beam from being incident on the sample, and/or a phenomenon occurs that ionization after the laser beam is incident on the sample is inhibited.

It is seen from the above results as a whole, in the case of the concentration conditions 3 to 4, droplets 5 on substrate 1 can be prevented from being fused, and a clear peak is observed in the mass spectrum. Therefore, the concentration of the surfactant in oil 6 on substrate 1 is preferably about 1 to 2% corresponding to the concentration conditions 3 to 4.

### [Humidity Conditions]

The humidity around substrate 1 during the fixation is, for example, a relative humidity of 75% or less, and preferably a relative humidity of 50% or less, however, the humidity is not limited to the above-specified ones. The relative humidity is, for example, the relative humidity of the atmosphere in a closed system (box). The humidity is adjusted by a humidity adjustment unit such as the humidity control unit (dehumidification) STU-1 available from AS ONE Corporation. The humidity is measured, for example, by the thermometer-hygrometer with external sensors (with internal and external temperature and humidity sensors) AD5648A available from A&D Company, Limited. More specifically, the humidity may be 75% or less as long as the coffee ring effect does not affect later use and/or analysis. For use and/or analysis where it is preferable that evaporation residues 51 remain more uniformly, the humidity is preferably 50% or less and, in this case, a lower humidity is a more preferable condition. It should be noted that the coffee ring effect generally refers to a phenomenon that, while a droplet is being dried, particles in the droplet move to the edge of the droplet to accumulate at the edge, resulting in a residue in the form of a ring. The coffee ring effect in the present example refers to a phenomenon that, while droplet 5 is being dried, a substance (such as a sample) in droplet 5 moves to the edge of the droplet and dries to solidify along the edge. In this way, evaporation residues 51 that can be analyzed and/or evaporation residues 51 suitable for analysis are generated, as illustrated exemplarily in the following.

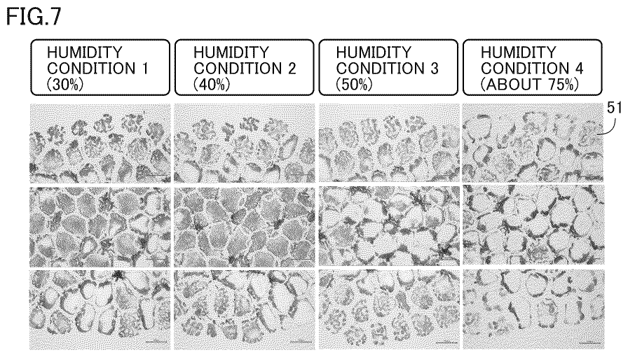

FIG. 7 is a diagram showing evaporation residues under different humidity conditions. In order to identify the position of droplet 5, water in which a red food coloring is dissolved is enclosed in droplet 5 of FIG. 7. In this way, the position where droplet 5 is fixed on substrate 1 is colored. Thus, a distribution of the positions where respective droplets 5 are fixed can be identified, based on a distribution of the color on substrate 1.

FIG. 7 shows evaporation residues 51 dried under the humidity conditions 1 to 4. Humidity conditions 1 to 4 herein correspond to conditions where the humidity around substrate 1 is 30%, 40%, 50%, and about 75%, respectively. For the humidity conditions 1 to 3, the humidity was adjusted by the humidity control unit (dehumidification) STU-1 of AS ONE Corporation. For the humidity condition 4, the humidity around substrate 1 was not particularly adjusted, resulting in a humidity of about 75%.

Other environmental conditions included a condition that the temperature was room temperature. The amount of first oil 6A in which droplets 5 were dispersed before being placed on substrate 1 was 1 μL, the concentration of the surfactant in first oil 6A was 1%, the amount of second oil 6B placed in advance on substrate 1 was 5 μL, and the concentration in second oil 6B was 1%. As each of first oil 6A and second oil 6B, oil prepared by diluting 2% (w/w) 008-FluoroSurfactant (RAN Biotechnologies) with fluorine oil was used.

Referring to FIG. 7, as the humidity during the fixation is lower, evaporation residues 51 isolated more clearly from each other are formed. On the contrary, as the humidity is higher, the boundary between evaporation residues 51 is vaguer.

Consequently, as the humidity during the fixation is lower, it is easier to identify the position corresponding to each evaporation residue 51. Under the low humidity condition, it is also possible to confirm that droplets 5 are not fused together during the fixation.

In addition, as the humidity during the fixation is lower, water in droplet 5 is dried at a higher speed, which hinders the coffee ring effect, so that droplet 5 is fixed in a state where the sample in the droplet extends uniformly. Referring to FIG. 7, as the humidity during the fixation is lower, the proportion of evaporation residues 51 colored uniformly in both the edge portion and the inner portion is higher, and the proportion of evaporation residues colored in the whole of the edge, rather than colored in only a part of the edge is higher. It is seen from the foregoing that the content of droplet 5 is distributed more uniformly as the humidity during the fixation is lower.

### [Pretreatment Method According to Modification]

It is also possible to perform gene analysis on a sample prepared by a pretreatment method according to an embodiment. In the following, pretreatment for gene analysis is described as a modification. Gene analysis corresponds to one example of “analysis of samples.”

A pretreatment method according to the modification may be performed by controller 9 controlling pretreatment device 81, or may be performed manually by a user.

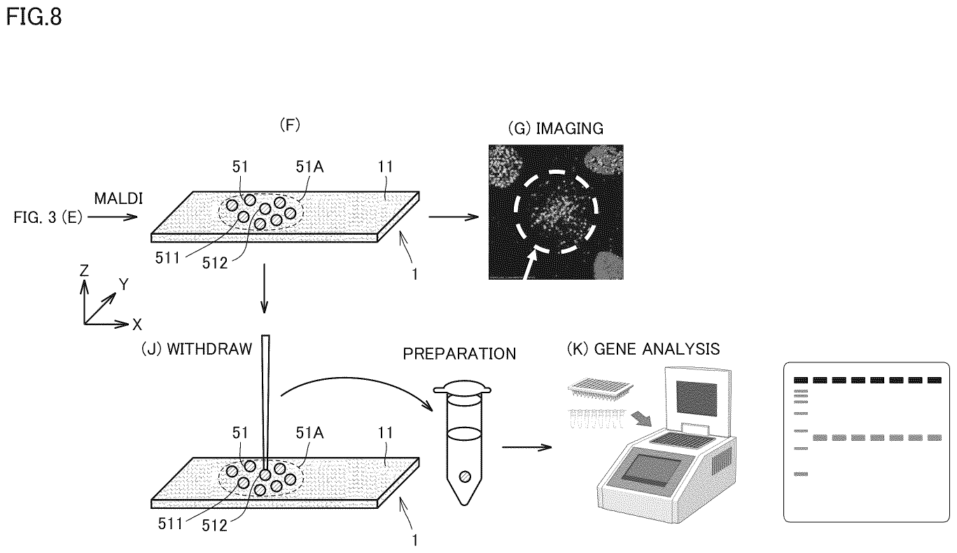

FIG. 8 is a diagram for illustrating pretreatment according to the modification. In the modification, the steps of (A) to (C) in FIG. 2 and (D) to (E) in FIG. 3 are similar to those of the embodiment, and are therefore not shown in FIG. 8. In FIG. 8, (J) shows a step peculiar to pretreatment for gene analysis. In FIG. 8, (K) shows the step of gene analysis.

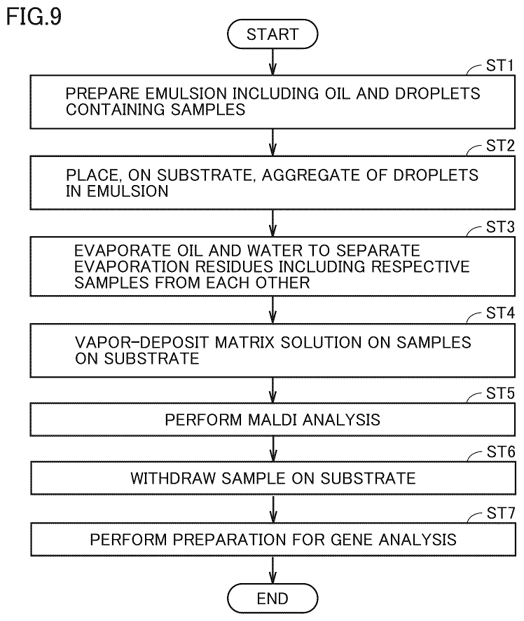

FIG. 9 is a flowchart showing an example of the pretreatment method according to the modification. Each step in FIG. 9 may be performed by controller 9 controlling pretreatment device 81, or may be performed manually by an analyst. In the following, pretreatment according to the modification is described following FIG. 9, with reference to FIG. 8 as appropriate.

ST1 to ST4 in FIG. 9 correspond to ST1 to ST4 in FIG. 4, respectively.

In ST5, MALDI analysis is performed on a sample prepared in ST4 in such a manner that enables the sample to undergo the MALDI analysis.

In ST6, a sample on substrate 1 is withdrawn. Specifically, for example, a predetermined evaporation residue on substrate 1 is scraped away by means of a capillary. In one example, the predetermined evaporation residue is an evaporation residue having a higher content of a predetermined substance than other evaporation residues, as found by MALDI analysis.

In ST7, preparation for gene analysis is performed for the withdrawn sample. More specifically, preparation for gene analysis is performed for at least one of the first and second samples on substrate 1. One example of the preparation for gene analysis includes mixing the withdrawn sample with a PCR (Polymerase chain reaction) solution. The PCR solution contains a substance necessary for PCR (the substance is a primer for amplifying a predetermined gene sequence, a thermostable DNA polymerase, or the like).

The sample having undergone the pretreatment of FIG. 8 can be subjected to gene analysis. For example, it is possible to separate the sample based on the size or analyze its sequence by electrophoresis.

In the example illustrated in FIG. 8, gene analysis can be performed on a sample having undergone MALDI analysis. Accordingly, multiple types of biological characteristics (e.g., proteins and genes) of a sample included in a predetermined evaporation residue can be revealed. For example, genes included in a predetermined evaporation residue having a higher content of a predetermined substance than other evaporation residues can be analyzed. Thus, it possible to search for a gene that causes a higher content of the predetermined substance.

In another example of the modification, ST6 and ST7 may be performed subsequently to ST1 to ST3, without performing ST4 to ST5. In other words, it is also possible to withdraw, by means of a capillary or the like, samples ((C) in FIG. 2) fixed in a state of being separated from each other up to ST3, and perform gene analysis on the samples.

According to the modification, it is possible to perform gene analysis on at least one of the samples in respective droplets fixed with a high throughput.

### EXAMPLES

One example of the embodiment and the modification is described in the following.

FIG. 10 is a diagram illustrating withdrawal of samples according to an example. The left chart and the right chart in FIG. 10 are respective photographs of a MALDI plate before and after the withdrawal, respectively where the photographs are taken from the side of substrate 1 (the surface 12 side) on which samples are not fixed. In the left chart, a plurality of evaporation residues (e.g., the evaporation residue indicated by reference numeral 34) and traces of a laser beam applied during MALDI analysis are shown on the MALDI plate. The right chart is the photograph taken after withdrawing the evaporation residue indicated by reference numeral 34 by means of a capillary indicated by reference numeral 32. The right chart shows a trace 36 left after scraping away the evaporation residue indicated by reference numeral 34.

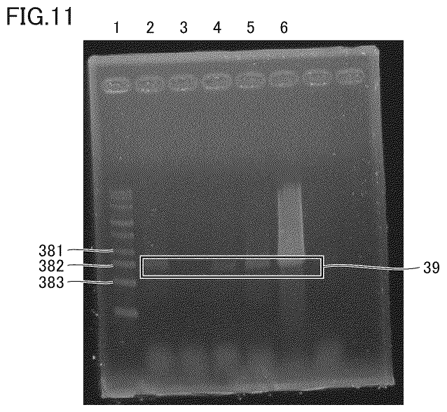

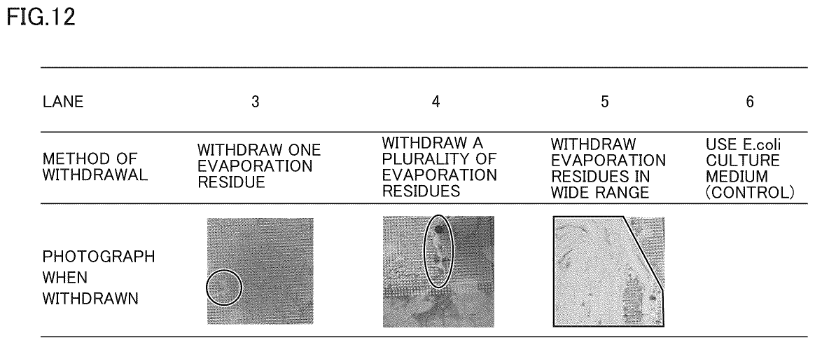

In the example, the pretreatment of FIG. 8 was performed on E. coli (Escherichia coli). Specifically, a plurality of droplets were generated from a bacterial medium in which E. coli was cultured, and the plurality of droplets were fixed on a substrate. After MALDI analysis was performed on evaporation residues generated on the substrate, the evaporation residues were withdrawn, subjected to preparation, and then subjected to PCR. As a primer for PCR, 16S rRNA gene universal primer was used. More specifically, 8F (a.k.a. 27F) was used as a forward primer and 1492R was used as a reverse primer. Accordingly, in the case where 16S rRNA of E. coli is contained in a sample, it is expected that a band of about 1500 bp (theoretically 1,483 bp) is detected by electrophoresis of PCR products. The results of actually performed electrophoresis are shown in FIGS. 11 to 12.

FIG. 11 is a diagram showing a photograph of a gel obtained by electrophoresis of a PCR product. FIG. 12 is a diagram showing a table illustrating a sample corresponding to each lane of the gel.

Referring to FIGS. 11 and 12, lane 1 shows the result of electrophoresis of size markers. As size markers, Perfect DNA® Markers, 0.5-12 kbp (Novagen) were used. Bands indicated by reference numerals 381, 382, and 383 correspond to 2000 bp, 1500 bp, and 1000 bp, respectively. In FIG. 11 a range of about 1500 bp corresponding to 16S rRNA is indicated by a frame 39.

Lane 3 shows the result of gene analysis of one evaporation residue withdrawn and subjected to preparation. Since no band is visible in lane 3, it is considered that the evaporation residue withdrawn from lane 3 was originally a droplet containing no E. coli.

Lane 4 shows the result of gene analysis of a plurality of evaporation residues withdrawn and subjected to preparation. In lane 4, a band corresponding to about 1500 bp is detected. Accordingly, it is considered that at least one of a plurality of droplets corresponding to the plurality of evaporation residues contained E. coli.

Lane 5 shows the result of gene analysis of evaporation residues in a wide range withdrawn together and subjected to preparation. In lane 5, the detected band corresponding to about 1500 bp is darker than that in lane 4. Therefore, it is considered that at least one of the plurality of droplets corresponding to the evaporation residues in the wide range contained E. coli.

Lane 6 shows the result of electrophoresis of a culture medium of E. coli subjected to PCR as control. In lane 6, the band including about 1500 bp is detected.

As seen from the above, according to the present example, DNA derived from E. coli was detected from the evaporation residue fixed on substrate 1, even after MALDI analysis was performed. Thus, it has been established that gene information of cells contained in droplets can be analyzed from the samples fixed on substrate 1, by the pretreatment methods according to the embodiment and the modification.

### [Aspects]

It should be understood by those skilled in the art that a plurality of exemplary embodiments as described above are specific examples of the following aspects.

(Clause 1) An analytical pretreatment method according to one aspect is a pretreatment method for analysis of samples, including:
preparing an emulsion including
   oil, and
   a first droplet and a second droplet being present in the oil and containing a first sample and a second sample respectively;
placing, on a substrate, an aggregate of the first droplet and the second droplet in the emulsion; and
evaporating the oil and water on the substrate to separate a first evaporation residue and a second evaporation residue from each other that include the first sample and the second sample respectively.

The analytical pretreatment method according to Clause 1 enables a high-throughput method to be provided for fixing samples in respective droplets in a state where the samples are separated from each other.

(Clause 2) In the analytical pretreatment method according to Clause 1, the first sample and the second sample are samples of the same type.

The analytical pretreatment method according to Clause 2 enables each of the first and second samples to be analyzed, and accordingly enables analysis of how multiple types of samples derived from cells of the same type are changed.

(Clause 3) In the analytical pretreatment method according to Clause 1 or 2, the first sample and the second sample are microorganisms, and the analytical pretreatment method further includes culturing the microorganisms in the emulsion.

The analytical pretreatment method according to Clause 3 enables microorganisms themselves in respective droplets and/or a predetermined metabolite generated from the microorganisms, to grow to a sufficient extent, and then undergo analysis.

(Clause 4) In the analytical pretreatment method according to any one of Clauses 1 to 3, environmental conditions under which the evaporating is performed include a concentration condition that a concentration of a surfactant in the oil on the substrate is within a predetermined concentration range, and/or a humidity condition that a humidity around the substrate is within a predetermined humidity range.

The analytical pretreatment method according to Clause 4 enables evaporation residues 51 that can be analyzed and/or evaporation residues 51 suitable for analysis to be generated.

(Clause 5) In the analytical pretreatment method according to Clause 4, the concentration range is 1% by mass or more and 4.5% by mass or less.

By the analytical pretreatment method according to Clause 5, evaporation residues 51 that can be analyzed are generated.

(Clause 6) In the analytical pretreatment method according to Clause 4 or 5, the humidity range is 75% or less.

By the analytical pretreatment method according to Clause 6, evaporation residues 51 that can be analyzed are generated.

(Clause 7) In the analytical pretreatment method according to any one of Clauses 1 to 6, the oil is equivalent to or higher in volatility than the water.

The analytical pretreatment method according to Clause 7 enables oil to be evaporated at a speed equivalent to or higher than the speed at which water is evaporated. As a result, it is possible to prevent droplets from being deformed due to the oil, while water is evaporated. Moreover, at the time water has been evaporated completely, oil has also been evaporated completely, and therefore, it does not occur that droplets are deformed due to remaining oil.

(Clause 8) In the analytical pretreatment method according to any one of Clauses 1 to 7, the first sample and the second sample each include a cell and/or a cell-derived substance.

The analytical pretreatment method according to Clause 8 enables analysis of components included in a droplet, and accordingly enables analysis of the cell itself (cell body, for example) or metabolites located inside and outside the cell.

(Clause 9) In the analytical pretreatment method according to any one of Clauses 1 to 8, the analysis is mass spectrometry using MALDI (Matrix-Assisted Laser Desorption) method, and the substrate is a sample plate for the mass spectrometry using MALDI method.

The analytical pretreatment method according to Clause 9 enables MALDI analysis to be performed on a sample having undergone the analytical pretreatment method, to thereby enable analysis of components in the sample. In particular, MALDI imaging can be conducted to thereby enable high-throughput analysis of each sample on the substrate.

(Clause 10) The analytical pretreatment method according to Clause 9 further includes vapor-depositing a matrix solution on the first sample and the second sample on the substrate, after the evaporating.

The analytical pretreatment method according to Clause 10 enables the matrix solution to be vapor-deposited easily and conveniently on each of the samples, even when the samples are dispersed irregularly on the MALDI plate.

(Clause 11) The analytical pretreatment method according to any one of Clauses 1 to 10 further includes performing preparation for gene analysis on at least one of the first sample and the second sample on the substrate, after the evaporating.

The analytical pretreatment method according to Clause 11 enables gene analysis to be performed on at least one of samples in respective droplets fixed with a high throughput.

While the embodiments of the present invention have been described, it should be construed that the embodiments disclosed herein are given by way of illustration in all respects, rather than by way of limitation. It is intended that the scope of the present invention is defined by claims, and encompasses all variations equivalent in meaning and scope to claims.

What is claimed is:

1. An analytical pretreatment method for analysis of samples, the analytical pretreatment method comprising:
preparing an emulsion comprising
oil, and
a first droplet and a second droplet being present in the oil and containing a first sample and a second sample respectively;
placing, on a substrate, an aggregate of the first droplet and the second droplet in the emulsion; and
evaporating the oil and water on the substrate to separate a first evaporation residue and a second evaporation residue from each other that include the first sample and the second sample respectively.

2. The analytical pretreatment method according to claim 1, wherein the first sample and the second sample are samples of the same type.

3. The analytical pretreatment method according to claim 1, wherein
the first sample and the second sample are microorganisms, and
the analytical pretreatment method further comprises culturing the microorganisms in the emulsion.

4. The analytical pretreatment method according to claim 1, wherein environmental conditions under which the evaporating is performed include a concentration condition that a concentration of a surfactant in the oil on the substrate is within a predetermined concentration range, and/or a humidity condition that a humidity around the substrate is within a predetermined humidity range.

5. The analytical pretreatment method according to claim 4, wherein the concentration range is 1% by mass or more and 4.5% by mass or less.

6. The analytical pretreatment method according to claim 4, wherein the humidity range is 75% or less.

7. The analytical pretreatment method according to claim 1, wherein the oil is equivalent to or higher in volatility than the water.

8. The analytical pretreatment method according to claim 1, wherein the first sample and the second sample each comprise a cell and/or a cell-derived substance.

9. The analytical pretreatment method according to claim 1, wherein
the analysis is mass spectrometry using MALDI (Matrix-Assisted Laser Desorption) method, and
the substrate is a sample plate for the mass spectrometry using MALDI method.

10. The analytical pretreatment method according to claim 9, further comprising vapor-depositing a matrix solution on the first sample and the second sample on the substrate, after the evaporating.

11. The analytical pretreatment method according to claim 1, further comprising performing preparation for gene analysis on at least one of the first sample and the second sample on the substrate, after the evaporating.
